(19) United States
(12) Patent Application Publication
(10) Pub. No.: US 2003/0134414 A1
(43) Pub. Date: Jul. 17, 2003

(54) Nerve growth assistance improvement

(76) Inventor: Ian Andrew Ferguson, Bedford Park (AU)

Correspondence Address: Akerman Senterfitt & Edison, P O Box 3188, West Palm Beach, FL, 33402-3188, US

(21) Appl. No.: 10/221,305

(22) PCT Filed: Mar. 12, 2001
(86) PCT No.: PCT/AU01/00268

(30) Foreign Application Priority Data
Mar. 10, 2000 (AU) PQ 6142

(51) Int. Cl. C12N 5/08

(52) U.S. Cl. 435/368; 435/404

(57) ABSTRACT

Material and method for promoting the re-growth of the CNS in mammals, including humans. This involves ligating a peripheral nerve, then excising the resulting material distal to the point of ligation (6) after a substantial delay. The nerve material (7), which is rich in vivo activated glial cells, is then finely minced (5) and in combination with a support matrix, and/or other nerve growth promoting materials (3), inserted into the spinal cord injury cavity (2) via syringe (4) so as to promote growth of the corticospinal tract axons (8) in the CNS (1).

### BACKGROUND OF THE INVENTION

### Technical Field

The present invention relates to materials and methods for effecting central nervous system nerve re-growth for mammals.

A number of people are injured each year where the injury causes traumatic spinal cord injury (SCI). While the incidence of SCI in the United States ranges from 28 to 50 per million, because most patients suffering SCI now survive, the prevalence of patients with chronic spinal cord injury is steadily growing and is estimated to be 183,000 to 230,000 in the United States alone.

Spinal cord injury resulting in damage to the CST results in paralysis below the site of injury. This paralysis is currently considered permanent because the corticospinal tract (CST) does not regrow past the injury site.

This invention is directed to methods and to materials which provides assistance in effecting nerve re-growth past an injury site within the central nervous system of mammals when the injury is in a chronic stage as compared to an acute stage.

An object of this invention then is to provide a material and a method of treatment facilitating regeneration of chronically injured nerves within the spinal cord sometime after the injury has occurred (i.e. chronic paralysis).

### DISCLOSURE OF THE INVENTION

One form of the invention can be said to reside in the treatment of spinal cord injury with a nerve growth promoting material having as an active agent, material derived from nerve tissue located outside the blood brain barrier that has had some of the functional connection between its nerve cell bodies and the remainder of this nerve tissue previously interrupted for a substantial time.

In preference, there is a targeted delivery of therapeutic agents to the chronically injured nerves by making use of a cavity which has been found to develop at the site of spinal cord injury as a location into which can be administered graft tissues or cells or therapeutic agents or pharmaceutical formulations. There is also described how to monitor the effects of the test therapeutic agents and pharmaceutical formulations chronically injured nerves by anatomical methods.

In one form the invention can be said to reside in a nerve growth promoting material having as an active agent, material derived from nerve material that has been separated from a nerve positioned outside the blood brain barrier but within a living mammalian body, which has had some of the functional connection between the nerve cell bodies and the nerve material previously interrupted for a substantial time.

In preference, the functional connection between the nerve cell bodies and the remainder of the nerve tissue will have been previously interrupted for a substantial period of time which is to ensure that there is a sufficient time for the active materials to be expressed sufficiently. This may be in preference as little as 1 day or through to as much or more than 60 days although 2 to 10 days is more preferred and approximately 7 days is found to be most preferred at this time. The interruption in preference is by ligation but other techniques are possible including severing, crushing, or chemical means causing axons to degenerate resulting in an abundance of in vivo activated glial cells that promote re-growth of the chronically injured central nervous system nerves.

In another preferred embodiment of the invention the nerve growth promoting material is and is administered in combination with other nerve growth promoting factors including neurotrophins such as nerve growth factor (NGF), brain-derived neurotrophic factor (BDNF), neurotrophin-3 (NT-3), neurotrophin-4 (NT-4), and/or members of the family of glial cell derived neurotrophic factor (GDNF), and/or other growth promoting molecules such as leukemia inhibitory factor (LIF) and/or those that act on receptors expressed by the CST such as the trkB and trkC neurotrophin receptors.

The heretofore mentioned methods are useful for the treatment of nerve damage to the central nervous system (CNS) associated with either physical or surgical trauma or other disorders that result in cavities or spaces within the CNS tissue. Example of interventions or disorders that can result in cavities, spaces or abscesses include: surgical removal of tumors or foreign tissue, traumatic injury or lesions of the tissues of the CNS, certain viral or bacterial infections of a central nervous system or neurodegenerative conditions such as Parkinson's disease or Alzheimer's disease.

In the above procedures, the nerve growth promoting material may be mixed with and administered when in combination with a physiological acceptable support matrix preferably gelling collagen or fibrin.

The invention can further be said in preference to reside in a pharmaceutical formulation comprising:

a nerve growth promoting material having as an active agent, material derived from nerve material that has been separated from a nerve located outside the blood brain barrier but within a living mammalian body, which has had at least some of the functional connection between the nerve cell bodies and the remainder of nerve material previously interrupted for a substantial time;

and, a physiological acceptable support matrix.

The material can have other agents that have been found to assist the action further.

The nerve material used in the above formulation can in preference be said to be a method of promoting nerve growth as above characterised in that the location of the cavity is adjacent nerve endings to be grown. In preference the method is further characterised in that the location of the cavity is adjacent nerve endings to be grown.

The nerve material in preference is derived from a nerve or nervous tissue located outside a blood brain barrier such as the sural nerve, saphenous nerve or olfactory epithelium. Alternatively or in addition, the in vivo activated glial cells may be derived from stem cells. In preference, but not essential, the graft tissues or cells will be obtained from the host to avoid immunological rejection.

Also provided is a description of how to target the delivery of the above pharmaceutical formulation or test graft tissues or cells and/or test therapeutic agents to the chronically injured nerves of the CNS by using the cavity or space which develops naturally, or without surgical intervention aimed at creating a cavity, at the site of spinal cord injury as a depot. By using the cavity as a depot for drug delivery and graft purposes, the cavity is transformed from being part of the problem (a physical barrier to CST regeneration) into part of the solution.

The present invention is directed at a nerve growth promoting material resulting from a nerve located outside the blood brain barrier that has had the functional flow of the axons interrupted therefrom after a substantial time for use for insertion into an injury cavity of a central nervous system in the vicinity of a chronically injured nerve the growth of which is to be promoted.

In another embodiment, the present invention is directed to a method of promoting re-growth of chronically injured nerves of the CNS which method includes the steps of interrupting the functional flow of the axons in a nerve or nervous tissue located outside the blood brain barrier but within a living mammalian body, separating the injured material after a substantial time, minutely dividing the material, combining said material with a physiologically acceptable support matrix, and inserting said mixture into a cavity in close proximity to a chronically injured nerve ending the growth of which is to be assisted within a central nervous system of a living mammalian body.

The present invention, in preference, is further related to a method of promoting re-growth of chronically injured nerves of the CNS which method includes the steps of interrupting the functional flow of the axons in a nerve in a living mammalian body, separating the injured material after a substantial time, minutely dividing the material, combining said material with a physiologically acceptable support matrix, and inserting said mixture into a cavity adjacent a chronically injured nerve ending the growth of which is to be assisted within a central nervous system and where there is grey matter for the nerve to grow through.

Furthermore, the present invention also provides, in preference, a method for assisting re-growth of chronically injured nerves of the CNS further characterised in that the material is inserted into a central nervous system which is of a body which either:

has identical DNA to the body from which the material was derived; or

which is of a body which is the same body from which the material was derived.

It is apparent that many modifications and variations of this invention as set forth may be made without departing from the spirit and scope of the present invention. The specific embodiments now described are given by way of example only and the invention is limited only by the terms of the appended claims.

### BRIEF DESCRIPTION OF THE DRAWINGS

FIG. 1A diagrammatic representation of a method for stimulating the regeneration of the chronically injured CST by administering an in vivo activated glial cell based formulation into the spinal cord injury cavity. Spinal cord 1, Spinal cord injury cavity 2; In vivo activated glial cell based formulation 3; Syringe 4; Finely minced nerve tissue 5;. Ligation 6; Peripheral nerve distal to the ligation 7; and Corticospinal tract axons 8.

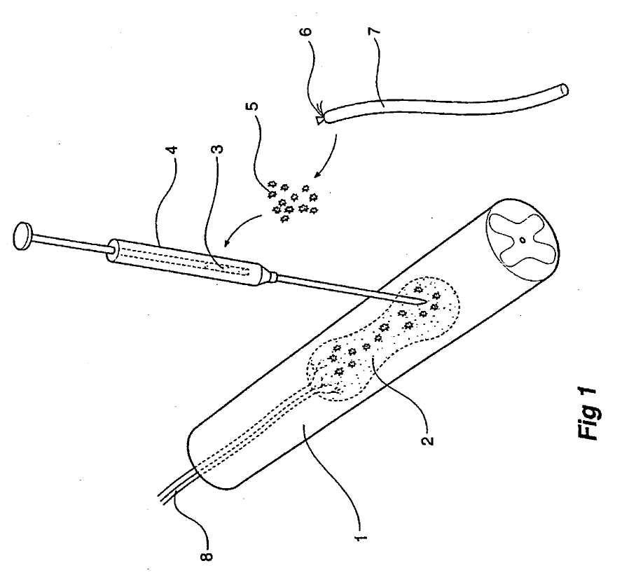

FIG. 2 Transplantation of preligated peripheral nerve tissue into the spinal cord injury cavity stimulates regeneration of the chronically injured CST. Animals generated as described in Example I. Data from representative animals that (A1-C1) received transplant of nerve segment, or (A2-C2) received minced nerve graft. Low (A) and higher power (B) magnification of HRP-filled GST growth cones with distribution of HRP reaction product caudal to the injury site (C). Animals processed for histology 2 weeks after grafting. Arrows indicate regenerating CST processes.

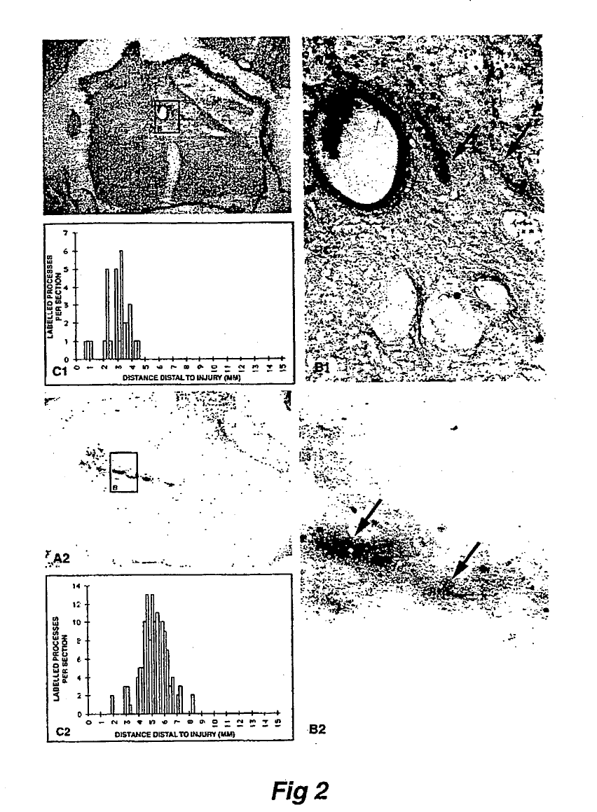

FIG. 3 Camera lucida and photomicrographs of horizontal section through injury site 12 months after transplant of preligated sural nerve into the cavity. Animals generated as described in Example 11. Graft tissue lost in histological processing. Arrows indicate anterograde labelled CST processes with BDA.

FIG. 4 Biotinylated dextran labelled CST processes (arrows) within the cavity wall and 5 mm caudal to rostral end of the cavity. Cavity filled with minced suspension of preligated sural nerve following infusion for 1 week with DMEM containing NT-3. An animal processed for histology at 2 weeks after grafting. Arrows indicate regenerating CST processes.

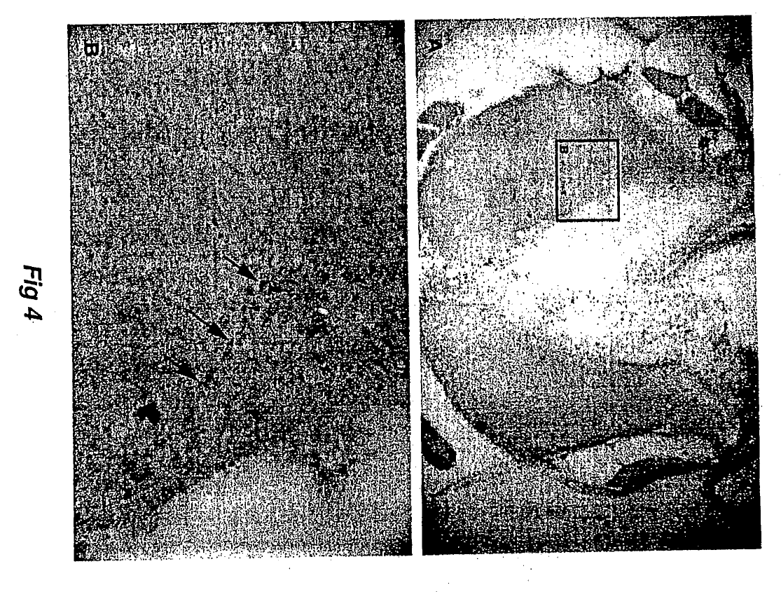

FIG. 5 Photomicrographs of cross-sections through the spinal cord injury cavities of two animals showing labelled CST sprouts which have grown out in response to administration over 1 week of a pharmaceutical formulation comprising NT-3 as described in Example IV. A and B are from an animal in which the CST was labelled using biotinylated dextran as the neuronal tracer; C and D are from an animal in which the CST was labelled using HRP/WGA-HRP as the neuronal tracer.

### BEST MODE FOR CARRYING OUT THE INVENTION

In humans, recovery of voluntary control over muscles paralysed due to spinal cord injury can require re-growth of the chronically injured CST. Before making use of the invention to treat human subjects, it is prudent to demonstrate that the invention is able to stimulate re-growth of the chronically injured CST in a suitable animal model. The following section instructs how to generate chronic spinal cord injured animals that may be used to demonstrate the invention.

Generation of Laboratory Rats with Chronic Spinal Cord Injury

A number of different methods for injuring the spinal cord may be used provided that the spinal cord injury results in axotomy of the CST and the formation of a cavity with preservation of sufficient central grey matter to allow CST re-growth through the cavity walls. Compared with many other methods of spinal cord injury such as compression injury, transection of the CST by partial dorsal hemisection can produce a very reproducible type of injury that results in less than profound permanent loss of hind-limb motor control. In contrast, compression injury often results in a spinal cord injury more analogous to that which occurs clinically in humans most frequently and this injury can be induced by a number of approaches including epidurally inflated balloons, clips and weight drop techniques each with their own advantages and disadvantages. For example, in the dynamic load or weight drop technique, the impact load can be varied to control the extent of spinal cord injury so that a significant correlation can be achieved between the size of the resulting cavity size and the severity of the loss of motor function. Provided that the method of inducing the spinal cord injury completely severs the medial CST of the rat and ensures that significant central grey tissue remains surrounding the cavity walls, either a partial dorsal hemisection and a weight drop approach may be used to generate spinal cord injured animals that may be used to demonstrate the invention. The procedures for creating these spinal cord injured animals are described below.

Methods

Adult female laboratory rats (Sprague Dawley strain, 6 weeks old) are anaesthetised by inhalational halothane (2-3% in oxygen) and fixed in a stereotaxic frame with nose cone. A keyhole approach is used to expose the spinal cord: Briefly, following a 2-3 cm midline incision of the cutaneous layers, the dorsal surface of vertebra T9 or T10 is exposed by using curved iris scissors to separate muscle layers and expose the bone. The spinal cord is exposed by removing the dorsal surface of the vertebra bone using curved micro-suture holders as bone rongeurs. This approach typically results in little bleeding and is completed within 10-15 minutes.

With the dorsal surface of the spinal cord exposed, a controlled injury to the spinal cord may be achieved by either of the two methods:

1. Spinal Cord Injury—Partial Dorsal Hemisection

To perform a partial dorsal hemisection, use of a stereotaxic knife is required to achieve reproducible cavity formation. While partial dorsal hemisection can be produced by hand, the depth and extent of injury can not be reproducible controlled as illustrated by the range of physical outcomes and variation in the size of the resulting cavities. A controlled and reproducible spinal cord injury is generated by a hemisection of the dorsal two-thirds of the spinal column using a razor blade attached to the pivot point of a cut down Vance micro-scissors fixed in the electrode holder of the stereotaxic frame. This semicircular incision severs the medial CST. Bleeding is stemmed and a piece of artificial dura (Dura-film, UpJohn; approx. 1.5×2.5 mm) is placed over the incision in the spinal cord. A piece of absorbable collagen matrix (DuoDerm, Squibb; approx. 2.5×2.5 mm) is placed in the bone/muscle cavity and 5/0 sutures used to close the separated muscles and cutaneous incision. The rat is then removed from the stereotaxic frame, weighed administered analgesic (Buprenorphine HCl, s.c., 80 mg/kg), and placed into a recovery cage with fresh bedding, food and water to recover from the effects of anaesthetic.

2. Spinal Cord Injury—Compression Injury

A controlled and reproducible spinal cord injury can also be generated by a transient compression of the spinal cord using a weight drop method. A specially designed teflon probe is lowered onto the surface of the exposed spinal cord. Transient compression of the spinal cord is achieved by releasing a 10 gm teflon weight that is suspended at known height above the spinal cord. The preferred height is 1.25 cm. This will result in a injury cavity of that is of sufficient size to completely sever the medial CST but not so large as to result in no central grey matter in the cavity wall—the CST fibres regrow through this spared central grey tissue. The teflon weight is allowed to fall under gravity 1.25 cm down a vertical rod and impact with the top of the probe resting on the surface of the spinal cord, thus transferring the impact energy into the spinal cord and briefly compressing the spinal cord. A piece of absorbable collagen matrix (DuoDerm, Squibb; approx. 2.5×2.5 mm) is placed in the bone/muscle cavity and 5/0 sutures used to close the separated muscles and cutaneous incision. The rat is then removed from the stereotaxic frame, weighed administered analgesic (Buprenorphine HCl, s.c., 80 mg/kg), and placed into a recovery cage with fresh bedding, food and water to recover from the effects of anaesthetic.

Effects of the Spinal Cord Injury and Post-Operative Care

Spinal cord injured rats are examined at least twice daily for the first three days and daily for the next 4 days. The daily examination involves observation of motor function on a flat surface and reflex testing and examination for signs of pain or distress (piloerection, vocalisation). Animals are then weighed, water consumption measured and returned to their cage. During the first 3 postoperative days, animals receive twice daily injections of analgesia (Buprenorphine HCl, s.c., 80 mg/kg). For post-operative days 4-7, animals received paracetamol in their drinking water. Using either spinal cord injury procedure, the spinal cord injury results in profound loss of hindlimb function during the spinal shock period that lasts approximately 3 days. This is followed by progressive recovery of hindlimb locomotor function and reflexes. By one week after the operation spinal cord injured rats exhibit nearly normal movement in the cage or on examination table. Unlike more severe spinal cord injuries, these spinal cord injury procedures described above do not normally cause permanent or severe loss of a normal bladder reflex. With this operation, animals typically do not show obvious signs of pain or distress (piloerection, vocalisation, etc). Histological studies show that by 3 weeks, these animals have a chronic spinal cord injury as evidenced by the presence of a well developed spinal cord injury cavity and complete severing of the medial CST. These animals may be used to test different cavity filling formulations for their ability to stimulate regrowth of the chronically injured CST.

Administration of Agents into the Spinal Cord Injury Cavity

Following spinal cord injury in humans and administration of anti-inflammatory agents such as methyl-prednisolone and possible surgical stabilisation of the vertebra at the injury site, the accepted clinical course is to allow the patient to stabilise, the inflammation to subside and then undertake a neurological examination to ascertain the loss of function and hence extent of injury. One consequence of this time course of treatment is that any invasive intervention is necessarily delayed for weeks or months after the paralysing injury until after there is no reasonable prospect of further recovery of function due to normal bodily processes. A second consequence is that f there is to be an improvement in function, the invasive intervention shall be capable of stimulating the now chronically injured nerves to commence re-growing past the injury site in the spinal cord.

It seems that in general workers in the field were not aware of the need to focus their investigations on identifying agents that are able to stimulate the chronically injured nerves to regeneration. At the time of invention, the dominant animal testing paradigm was one in which the experimental therapeutic agent (e.g. graft tissue) was administered into the spinal cord injury site at the time of injury. While these studies demonstrate the capacity of acutely injured nerves to regenerate, they do not demonstrate (i) whether such injured nerves retain this regenerative capacity after the injury; and, (ii) how to stimulate nerve regeneration in a manner which might be developed into a clinically useful procedure.

Delaying administration of therapeutic agents can be expected to have a number of distinct advantages. For example, where therapeutic agents are administered into the injury site shortly after injury, they can be expected to b e in an anoxic environment and exposed to secondary destructive processes (e.g., phagocytosis) as a result of the inflammatory process. Consequently, by using the spinal cord injury cavity for drug delivery purposes rather man directly into the injury site shortly after the injury, the potentially beneficial putative therapeutic agents or graft tissues can be more easily observed.

The following section describes simple methods for administering therapeutic agents into the naturally occurring spinal cord injury cavity and for monitoring the efficacy of these test agents in stimulating the chronically injured CST to commence re-growing past the spinal cord injury site. Also described is a simple formulation that makes use of the peripheral nerve tissue from the spinal cord injured subject and how to use it to stimulate the chronically injured CST to regenerate by administering it weeks or months after the paralysing injury.

Different procedures comprising different steps can be used to deliver the therapeutic agents into the spinal cord injury cavity. Because each injury is unique, it is not possible to prescribe one approach as being better than another in all cases. In human subjects, it will be helpful to gather as much information about the injury site and the cavity itself by supplementing the neurological assessment with information obtained by making use of imaging techniques such as magnetic resonance imaging and/or computerised tomography. To demonstrate the embodiment it is critical that substantial central grey tissue remains surrounding the cavity because animal experiments demonstrate that it is through this that the re-growing CST axons must grow.

Where a human patient is assessed as being likely to benefit from administration of therapeutic agents into the cavity, it will be important to consider which is a preferred method of administering the therapeutic agent. This may need to be done at the time of surgical access to the cavity and in performing this operation, the guiding principal to be used in gaining access to the spinal cord and in deciding what approach to use for administration will be to minimise the risk of creating secondary injury to the spinal cord and its delicate tracts. Keeping this principle in mind while operating will minimise the possibility that the intervention may actually make the patient worse in terms of function.

As demonstrated within using animal studies, administration of the therapeutic agents into the cavity will be by surgical incision to transplant tissue or injection via catheter. Using surgical incision, the underlying cerebrospinal fluid filled cavity is exposed by careful dorsal midline incision and the tissue inserted. This is practical because no nerve fibre pathways of consequence cross the midline at the dorsal funiculi. Alternatively, a catheter may be inserted at the midline into the underlying cavity. Entry of the catheter end into the cavity may be judged by the flow of cerebrospinal fluid out of the catheter. The choice of type of catheter and its diameter and angle of insertion will be determined by factors unique to the actual injury and consideration of the therapeutic agent to be delivered through the catheter into the cavity. In general, the catheter will be as narrow as possible but still of large enough bore to enable delivery of the therapeutic agent through the catheter and into the cavity at an acceptable rate (e.g. before the formulation gels).

With access to the cavity gained and an ability to administer the therapeutic agent enabled, a next step involves administration of the therapeutic agent. The choice and preparation of the therapeutic agent is described in more detail below. While more solid tissue for transplant may be grafted into the cavity, requiring cavity access by midline incision, preference is for an injectable formulation that may be delivered through a catheter into the cavity. One advantage of fitting an in-dwelling catheter into the cavity is that the catheter may be connected to a suitable delivery device such as pump. This can enable therapeutic agents such as nerve growth stimulating factors to be infused directly into the cavity over a period of time after the completion of operation to insert the catheter into the cavity. The same catheter may then be later used as a conduit for delivering into the cavity an injectable therapeutic graft tissue such as described in more detail below. That is, the pump is removed and a syringe is attached and used to deliver into the cavity a gelling matrix containing therapeutic cells, before the catheter is then carefully withdrawn from the cavity.

It will be apparent that before administering a therapeutic agent into the spinal cord injury cavity of a human patient it will be desirable to first test the therapeutic agent for ability to stimulate re-growth of the CST in an spinal cord injury animal model such as those described above. Described below is a two step procedure that can be used to administer therapeutic formulations into the cavity of spinal cord injured rats. The first step involves anaesthetising the rat to ligate the sural nerve and, without recovering from anaesthetic, to insert a catheter into the spinal cord injury cavity attached to an infusion pump to administer nerve growth stimulating factors while allowing the in vivo activation of the glial cells in the ligated sural nerve. The second step takes place 1 week later and involves removal of the ligated sural nerve, its use to prepare an injectable in vivo activated glial cell based formulation and its injection via the pump catheter into the spinal cord injury cavity. It should be noted that there is no absolute requirement to infuse nerve growth stimulatory factors into the spinal cord injury cavity prior to administration of the in vivo activated glial cell based formulation. Also, there is no absolute requirement to insert a catheter for use in administering the in vivo activated glial cell based formulation into the spinal cord injury cavity. This formulation can be injected by using an appropriate syringe and needle such as a 27 gauge needle fitted to a 10 μl Hamilton syringe. These procedures can be appropriately adapted for use in humans.

By 14 days after a spinal cord injury performed as described above, a well defined CSF filled cavity will have formed at the injury site. At this time, or after, the animal is anaesthetised with inhalational Halothane (2-3%) and fixed in a sterotaxic frame with nose cone. To provide a source of in vivo activated glial cell tissue for preparation of the injectable formulation 1 week later, the sural nerve is ligated. In this procedure, the sural nerve is exposed via a 1 cm incision in the lower left thigh. A 5/0 suture is used to ligate the nerve and to close its origin as a branch of the sciatic nerve. The incision is then closed using sutures.

While the animal is still under anaesthetic, a catheter can be inserted into the spinal cord injury cavity to be used 1 week later for administering the in vivo activated glial cell formulation into the spinal cord injury cavity and, in the meantime, to infuse nerve growth promoting factors into the cavity. This requires that the previous incisions are opened and piece of absorbable collagen matrix placed above the spinal cord injury is removed to expose the transparent dura film insert overlying a cavity of approx. 1.5-3 mm³. The dura film insert is carefully lifted off. A thin walled silastic tube of internal diameter less than 0.5 mm and prefilled with a physiologically acceptable solution such as Dubelco's Modified Eagles Medium (DMEM) is inserted into the cavity through a small incision in the dorsal surface and fixed in place with 10/0 sutures. The catheter tube is then attached to an appropriate continuous infusion devise such as an Alzet mini-osmotic pump and the muscle layers closed with 5/0 sutures. The mini-osmotic pump, which has been pre-filled with sterile DMEM containing appropriate nerve growth stimulating factors such as NT-3 (50 μg/ml), is inserted in a pocket below the skin formed by using blunt scissors to separate the muscle layer from the overlying cutaneous layer in the lower abdominal region. The substances to be infused into the spinal cord injury cavity will first be tested in tissue culture experiments to ensure they are non-toxic and to confirm sterility. The incision is then closed with 5/0 sutures.

The rat is then removed from the stereotaxic frame, weighed, and placed into a recovery cage with fresh bedding, food and water to recover from the effects of anaesthetic.

Effects of the Operation and Post-Operative Care

Appropriately performed the Step 1 procedures do not adversely affect the animals. Animals receive post-operative care appropriate to this type of surgical intervention such as a single injection of Buprenorphine HCl, s.c., 80 mg/kg.

Step 2—Preparation and Administration of in vivo Activated Glial Cell Formulation into the Spinal Cord Injury Cavity

One week after ligation of the sural nerve, the spinal cord injured rats are anaesthetised by inhalational halothane (2-3% in oxygen) and fixed in a stereotaxic frame with nose cone. If the animal was receiving an infusion of the nerve growth promoting substances substance(s), the mini-osmotic pump is removed and the catheter cut leaving a few mm projecting out of the muscle layer. Otherwise, the procedure described in Step 1 is followed to expose the spinal cord injury cavity. Then the incision overlying the sural nerve ligation is opened and a 1 cm segment of sural nerve below the ligation is removed to a 1.5 ml microfuge tube. One hundred microliters of ice cold sterile DMEM is added and the nerve is finely minced using micro-scissors. Mincing continues until the nerve tissue is sufficiently finely divided that is can be drawn up through a 27 gauge needle. The nerve tissue suspension is then briefly centrifuged (about 30 seconds in an Eppendorf microfuge) to pellet the tissue fragments. All the supernatant is removed, and 10 μl of DMEM is then added and a suspension recreated by brief trituration. The tube containing the minced nerve tissue returned onto ice. Also on ice and prepared beforehand will be (i) a microfuge tube containing 20 μg each of the nerve growth stimulating factors BDNF, NT-3 and GDNF in lyophilised form; and, (ii) another tube containing 3.75 mg/ml of sterile Type 1 rat tail collagen dissolved in 0.2% acetic acid. Immediately before injection into the cavity, 10 μl of collagen solution is added to the tube containing lyophilised nerve growth stimulating factors to bring them into solution. The collagen solution now containing nerve growth stimulating factors is then be added to the tube containing the minced nerve tissue, triturated briefly, and taken up into the ice-cold 10 _I Hamilton syringe. The needle of the Hamilton syringe is then fitted to the catheter previously inserted into the SCI cavity and the cavity filled with 1-3 μl of the gelling in vivo activated glial cell formulation. Alternatively, if a catheter is not fitted, the needle of the Hamilton syringe is attached to the electrode arm of the stereotaxic frame and the needle tip carefully inserted directly into the SCI cavity and the formulation administered. To facilitate accurate placement in the cavity, the needle tip should not be of the angled bevel type. In depressing the syringe plunger, care is taken to not forcefully deliver the solution into the cavity and so reduce the risk of traumatising the spinal cord. On the other hand, the syringe plunger should not be depressed so slowly as to allow sufficient time for the gelling collagen solution to warm and start to gel. At the appropriate rate of delivery, 1-3 μl can be delivered within 10 to 30 seconds. The cavity is filled when some of the formulation can be observed to ooze out of the spinal cord around the needle or catheter. The spinal cord and injury site is then examined to confirm good cavity filling and absence of odema (a sign of secondary trauma). A fresh piece of dura film is laid over the spinal cord injury site, and the incisions closed with sutures, and animal removed from the stereotaxic frame, weighed, and placed into a clean cage with fresh bedding, food and water to recover from the effects of anaesthetic.

Effects of the Operation and Post-Operative Care

Appropriately performed, the administration of therapeutic agents does not adversely affect the animals. Animals receive post-operative care appropriate to this type of surgical intervention such as a single injection of Buprenorphine HCl, s.c., 80 mg/kg. As described in more detail below, no rapid marked improvement in motor function should be expected given that these animals already exhibit nearly normal locomotion on flat surfaces.

Choice of Graft Tissuses or Cells

To be useful for treating humans with paralysis due to damaged CST, it is useful to have prior knowledge from animal studies that a particular therapeutic agent to be administered into the cavity can be expected to stimulate regeneration of the chronically injured CST when administered into the human subject's spinal cord injury cavity. While there has been no systematic testing of a wide range of possible therapeutic substances, published studies indicate that the CST in the acutely injured state, can not easily be stimulated to regenerate. Indeed, the CST is unlikely to regenerate even following attempts to reconstitute the spinal cord environment prior to injury. For example, there is no evidence that transplanting foetal spinal cord into the injury site to fill the cavity and reconstitute the spinal cord's cellular components can stimulate re-growth of the chronically injured CST. This is despite of the fact that such foetal spinal cord tissue appears to integrate well into the injured spinal cord and tracer studies indicate that short-range intersegmental circuitry in the injured adult rat spinal cord stimulated.

While it is possible to identify therapeutic agents by forming a hypothesis and then testing it (using the procedures described above), to make informed decisions about the potential therapeutic benefit of the test therapeutic agent, t is also important to include a positive control. Disclosed above is a therapeutic agent formulation that can be used a positive control in animal model studies aimed at discovering or developing further improved therapeutic formulations for use in human spinal cord injured subjects. We demonstrate herein and below a process for preparing a formulation comprising as a primary component, in vivo activated glial cells that can stimulate the chronically injured CST to regenerate.

A preferred but not the only source of these in vivo activated glial cells is the peripheral nerve tissue of the spinal cord injured patient. The primary cellular component of peripheral nerve tissue is the Schwann cell. The Schwann cell exhibits two distinct phenotypes depending on whether it is in contact with functioning nerve axons or whether is it axon deprived state. Normally, the Schwann cell is associated with functional axons and contributes myelin for facilitating electrical transmission along axons. In this form, the Schwann cell does not stimulate axonal sprouting and re-growth as evidenced by the relative lack of axonal sprouting and outgrowth that can be observed in an intact and normally functioning peripheral nerve. This contrasts with the Schwann cell that is deprived of functional contact by a variety of methods, most commonly, injury to the nerve. Injury to the nerve severs the axonal connection between the nerve cell body and the tissue (such as a muscle) that the nerve was previously innervating. The nerve axon, away from the nerve cell body and below the injury site, degenerates. This deprives the Schwann cells below the nerve injury of contact with functional nerve axons and, over the course of a few days, the Schwann cells change their phenotype in response. The phenotype of the Schwann cells changes so that they can positively stimulate and facilitate the re-growth of the injured nerve axons back down and along these activated Schwann cells to reinnervate the tissues they innervated prior to the nerve injury. We demonstrate that a nerve graft formulation comprising these in vivo activated Schwann cells, when it is introduced into the spinal cord injury site, is able to stimulate the chronically injured CST axons to start re-growing into the tissues it innervated prior to the injury.

While it is possible to deprive the Schwann cells of contact with functional axons by physically removing the peripheral nerve from the body and incubating it in tissue culture, typically as dissociated cells, this is not recommended for the invention. By removing the peripheral nerve into tissue culture, the nerve tissue is isolated from the body's normal injury response processes. For example, it is unable to benefit from the invasion of macrophages and other phagocytic cells that remove degenerating nerve axons and myelin. Also, the normal physiological response to injury involves complex changes in the local environment in terms of changes in the levels of various cytokines and growth factors and removing the peripheral nerve from this environment deprives the Schwann cells of their effects. Furthermore, it is possible that in addition to the Schwann cells, the invading macrophages, altered levels of cytokines and growth factors may also have positive and beneficial effects in stimulating the chronically injured CST to regenerate. Thus, by administering into the cavity, a formulation comprising peripheral nerve that has been axotomised and allowed to remain in vivo is likely to contain a much more complex mixture of cells and growth stimulating factors that a formulation prepared using fresh peripheral nerve tissue or Schwann cells passaged through tissue culture.

With this in mind, it will be apparent that almost any peripheral nerve tissue might be used to prepare the formulation, provided removal of that tissue is not going to unacceptably compromise the health of the patient. While the peripheral nerve tissue may come from a donor that is not the spinal cord injured patient, the preferred tissue will be genetically identical to the recipient That is, the tissue will come from spinal cord injured patient or from an identical twin or from stem cells prepared or taken from the spinal cord injured patient or engineered or modified so that tissue rejection or graft vs host disease is avoided.

The preferred source of peripheral nerve tissue for preparation of the formulation will be a peripheral nerve tissue that is widely accepted by neurosurgeons as being useful for peripheral nerve grafting or reconstruction. Nerves such as the sural nerve or the saphenous nerve are examples of such peripheral nerves. They are pure sensory nerves and their removal does not normally compromise the health of the patient. For use in the invention, one or both of these nerves can be surgically tied off one week before the preparation of the formulation for administration into the spinal cord injury cavity.

While the focus of the description of the invention is primarily on use of peripheral nerve tissue as a source of glial cell used to prepare the formulation, workers in the field will be aware that olfactory ensheathing glia that have been deprived of axonal contact by using in vitro tissue culture passaging techniques may also be used to prepare the formulation. There exists prior art to demonstrate that acutely injured CST can be stimulated to regenerate b y transplant into the injury site of olfactory ensheathing glia prepared from the olfactory bulb within the brain and hence within the blood brain barrier. The fact that olfactory ensheathing glia are routinely harvested from the brains of other animals constitutes evidence that workers in the field are not aware is that olfactory epithelium is an alternative source of olfactory ensheathing glia that is located outside the blood brain barrier and hence can be safely harvested by biopsy punch methods via the nose and that these glial cells can be obtained from the spinal cord injured patient thereby avoiding the risk of graft vs host disease. Furthermore, it is also not obvious to workers in the field of spinal cord injury that olfactory ensheathing glia can be deprived of contact with the axons that make up the olfactory nerves, by a non-surgical method. In this non-surgical method, a solution containing sufficient concentration (approximately 2% ZnCl₂) is applied to the olfactory epithelium, via the nose. The zinc ions cause the olfactory receptor neurons to degenerate thereby depriving the olfactory ensheathing glia in the olfactory epithelium of contact with functional axons and activating them for use in preparing an in vivo activated glial cell based formulation described herein. Thus, to prepare this n vivo activated glial cell based formulation comprising olfactory ensheathing glia, a 2% ZnCl₂ solution is applied by nose drops to the donor patient's olfactory epithelium. One week later, a biopsy punch is used to harvest the axotomised olfactory ensheathing glia and used to prepare the in vivo activated glial cell based formulation.

Choice of Graft Matrix

To enable injection and cavity filling, the in vivo activated glial cell based formulation will include components that will cause the formulation to gel after delivery into the cavity. This gel provides a supportive extracellular matrix for the activated glia and other cells delivered into the cavity. The gel also can function to slow the quick release of growth stimulatory molecules derived from the activated glial cell tissue source or that may be added into the formulation mixture prior to injection.

The preferred component to create a gel is collagen. The collagen will remain as a solution where a collagen solution is maintained at acid pH and on ice. When the pH is neutralised and the solution is warmed to body temperature, the individual collagen molecules in solution self-assemble into long fibrils that comprise the gel matrix. The invention instructs that the collagen solution is maintained under acidic conditions and on ice and only mixed with the activated glial cell preparation immediately prior to injection. This may be facilitated by making use of a two syringe injection approach where the collagen solution and the activated glial cell preparation are in separate syringes and the mixing takes place in the catheter tube as the formulation is injected.

A second useful gelling component is fibrinogen. Fibrinogen is the protein found in blood that causes it to gel or clot following enzymatic conversion to fibrin by the enzyme thrombin. Thus, either a purified fibrinogen solution or plasma may be used to generate the gelling matrix. The source of the plasma can be the spinal cord injured patient and will contain an appropriate anticoagulant such as trisodium citrate. This plasma can be obtained by mixing 9 volumes of freshly drawn blood with 1 volume of 3.8% trisodium citrate followed by centrifugation to separate blood cells from the plasma. To catalyse the gelling reaction, an appropriate amount of thrombin can be added immediately before injection. The amount of thrombin to be added can be determined experimentally to ensure that the fibrinogen containing solution will not gel before filling the cavity. As with the preparation of a gelling collagen matrix, use of a two syringe injection approach can minimise the risk that the solution will gel prematurely.

Choice of Therapeutic Agents for Administration

Animal model studies demonstrate that certain nerve growth stimulatory molecules, such as NT-3, can be added to the injectable formulation to stimulate robust sprouting of the chronically injured CST processes. The preferred or minimum preferred additives are potent nerve growth stimulatory molecules that have been demonstrated to act directly on the CST or upper motor neurons. These molecules include NT-3, BDNF and GDNF. Prior art studies using acutely injured CST animal models demonstrate that other molecules such as nerve growth factor (NGF) or leukemia inhibitory factor (LIF) may also be expected to enhance CST regenerative response. Other molecules may be included in the formulation with beneficial effects. As one example, acutely injured CST animal model studies indicate that certain inhibitors, such as antibodies that bind to and neutralise neurite inhibitory molecules such as NoGo, may be added to provide a robust or enhanced regeneration of the chronically injured CST. As another example, molecules include vascular endothelial growth factor (VEGF) that can be added to stimulate rapid in-growth of blood vessels and hence provide nutrient support for the cells delivered into the cavity. The beneficial effects of such additive molecules may be extended over time where a gene therapy approach is used to introduce into the transplanted cells the genetic instructions needed to sustain production and release of useful molecules.

Monitoring the Effect of Test Agents

As studies with rat spinal cord injury animal models demonstrate, use of the invention stimulates the chronically injured CST to regrow past the injury site in the spinal cord. As primary goal of stimulating CST regeneration is to allow recovery of at least partial voluntary control over the paralysed muscles below the spinal cord injury site, the question follows: Does use of the invention result at least partial recovery of lost voluntary control in mammals?

A key and important desired outcome of a chronic spinal cord injury treatment is that the chronically injured CST will have been stimulated to regenerate past the injury site and reconnect functionally with target spinal motor neurons below the injury site.

Although there are some fundamental differences between rat and other mammalian anatomy, the use of rats provides a readily accessible spinal cord injury animal model.

The CST is of relatively minor importance in the rat because motor control in such animals is dependent on spinal tracts that have their origins in sub-cortical regions of the brain (principally the reticular formation of the medulla and pons and the red nucleus in the mid-brain that give rise to the reticulospinal and rubrospinal tracts within the spinal cord, respectively). Indeed, by making use of the spinal cord injury procedures described above, it is possible to lesion the medial CST in the rat relatively selectively and this injury does not cause overt loss of hindlimb motor function after the rats recover from the spinal shock period.

Thus, in the rat model, evidence of recovery of previously paralysed hindlimb function does not necessarily indicate return of CST function.

There is, however, an absence of reliable methods to monitor at a functional level any recovery of the CST function in the rat and so this forces a reliance on anatomical measurements of CST regeneration.

Fortunately, acute spinal cord injury studies in rats demonstrate that the CST does not regenerate as easily as the reticulospinal and rubrospinal tracts.

Given this, when CST regeneration is observed, it is reasonable to assume that there will have been regeneration of injured reticulospinal and rubrospinal tracts. Indeed, studies using acute rat spinal cord injury animal models indicate that once the CST can be stimulated to grow past the injury site, without further intervention the regenerating CST axons will grow to and reconnect with their appropriate targets resulting in partial but significant recovery of function. For example, infusion of the monoclonal antibody IN-1 resulted in almost complete recovery of the lost contact placing response. In the contact placing response, which is dependent on the integrity of the CST, low threshold hair or light skin contact of the foot stimulates the animal to lift its limb and place it on the surface for support. Similarly, transplantation of myelin stimulated macrophages into the spinal cord injury site stimulates CST regeneration leading to partial recovery of cortical stimulation of myoelectric evoked potentials. Therefore, there is evidence that once the regenerating CST grows beyond the injury site, it can extend for many centimeters in the central grey and make functional reconnections resulting in partial restoration of lower limb function.

From these considerations, anatomical evidence that the invention is able to stimulate the chronically injured CST to regrow past the injury site in at spinal cord injury models can be used to gauge the potential therapeutic benefits of administering the inventive formulation into humans with spinal cord injuries of the appropriate type. To monitor whether or not a formulation administered into the spinal cord injury cavity in a rat has been able to stimulate the chronically injured CST to regenerate, the following neuroanatomical tract tracing procedure is recommended:

Administration of Neuronal Tracers

Two weeks before the planned euthanasia of the animal, the rat will receive tracer injection to anterogradely label the CST and their re-growing processes in the spinal cord. Briefly, the rat is anaesthetised by inhalational halothane and fixed in a stereotaxic frame. A 1 cm midline incision of the scalp is used to expose the underlying skull bone. A total of 6 small (1 mm dia.) burr holes are drilled through the skull overlying the motor cortex (3 holes over the left motor cortex and 3 over the right motor cortex). The stereotaxic coordinates are bregma: 0.0 to −4.0 mm; lateral: 1.5 mm. To apply the neuronal tracer, small (approx. 0.2 mm dia.) globes of dried biotinylated dextran (Molecular Probes) solution on the end of micropins are inserted through a small incision in the aura into the motor cortex to a depth of 0.5 mm. The micropins are left in place for 3-4 min to allow the biotinylated dextran solution to soften and detach from the end of the micropin. The micropins are then withdrawn leaving the tracer solution in the motor cortex. An alternative method of administering the neuronal tracer as a 10% solution of biotinylated dextran makes use of fine glass pipettes of tip diameter approximately 50 μm. The glass pipette is back-filled with the neuronal tracer solution by using a syringe attached b y tubing to the glass pipette. The tip of the glass pipette is then lowered into the motor cortex using the stereotaxic coordinates described above and approximately 700 nl of neuronal tracer solution delivered into the cortex at each site by pressure injection. Following administration of the neuronal tracer, the incision is then closed with 5/0 sutures and the animal removed from the stereotaxic frame and allowed to recover from the anaesthetic.

Histological Analysis

Two weeks after administration of the neuronal tracer, the animals are killed with an overdose of Nembutal and perfusion fixed using 4% paraformaldehyde, 0.5% glutaraldehyde in 0.1 M phosphate buffer, pH 7.3. The whole brain and spinal cord was dissected out, post-fixed overnight then cryoprotected in 30% sucrose in 0.1 M phosphate buffer, pH 7.3 for 24 hours. The whole brain and spinal cord was frozen and 50 μm sections cut and thaw mounted on slides to preserve the organisation of the whole CNS including anterogradely labelled CST and injury site. The presence of biotinylated dextran in axons can be visualised by first applying streptavidine-HRP conjugate followed by visualisation using tetramethylbenzidine (TMB) as described in the technical literature. The slides can be examined using light microscopy and photomicrographs and camera lucida drawing made to provide documentary evidence of CST regeneration.

Theoretical Use in the Clinic

In vivo activated glial cell based formulations may be administered to patients with chronic spinal cord injury to stimulate regeneration of the chronically lesioned CST. In its preferred form, the in vivo activated glial cell based formulation will be injected via catheter into the spinal cord injury cavity of a suitable patient. The surgical procedure of catheter insertion into the cavity makes use of a frequently used treatment for a patients with syringomyelia. In patients with syringomyelia the spinal cord injury cavity progressively increases in size leading to progressive loss of further function. A common treatment for this disorder involves the insertion of a catheter into the cavity with the aim of releasing cerebrospinal fluid and relieving internal pressure. A recent report of an experimental treatment for syringomyelia describes administration of embryonic spinal cord tissue dissected from aborted foetuses into the cavity to reduce the rate of growth in the size of the cavity. That study demonstrated that the procedure of administering tissue into the spinal cord injury cavity does not compromise patient safety.

The preferred sequence of steps involved using this invention in humans are:

1. Selection of spinal cord injured patients with good potential to benefit from administration of the treatment, for example, by virtue of the cavity having continuous significant spared central grey tissue;

2. In vivo activation of the patient's glial cells by, for example, surgical ligation of selected peripheral nerve(s) and allowing the glial cells to activate in vivo;

3. Removal of the tissue containing the in vivo activated glial cells and use to prepare an injectable activated glial cell based formulation that also includes, for example, nerve growth stimulating factors and gelling collagen;

4. Administration of the activated glial cell based formulation into the spinal cord injury cavity, preferably by injection via catheter; and,

5. Monitoring the recovery of patient and effects of the treatment.

As a practical example, again using procedures that work when used to treat rats with chronic spinal cord injuries, the treatment of spinal cord injury will preferably involve two teams, one team responsible for the medical care and surgical treatment of the patient, and the other responsible for preparation of the activated glial cell based formulation. In the operating theatre at the time of treatment, the surgical team is responsible for accessing the spinal cord injury cavity, insertion of a catheter and removal of the tissue containing the in vivo activated glial cells from the patient. This tissue is then transferred to the second team who, under sterile and aseptic conditions, then uses that tissue to prepare an activated glial cell based formulation which is taken up into a syringe ready for injection. The syringe containing the injectable activated glial cell based formulation is then passed to the surgical team for injection into the patient's spinal cord injury cavity. The medical team is responsible for monitoring the effectiveness of the treatment and other aspects of medical care of the patient. In its simplest form, the team responsible for the preparation of the injectable activated glial cell formulation can comprise a single individual skilled and practiced in the procedure described within for preparing injectable activated glial cell based formulations from in vivo activated glial cell containing tissue.

Below we describe experiments conducted in rats (n>50) with chronic spinal cord injuries that provide support of principal data and evidence establishing that the in vivo activated glial cell based formulation is able to stimulate the chronically injured CST to regenerate.

### EXAMPLE I

Grafting of Pre-Ligated Peripheral Nerve Segments or Minced Nerve into Spinal Cord Injury Cavity and Neuronal Tracing Using HRP/WGA-HRP

Adult female rats (6 weeks old; 15-200 g) were anaesthetised by inhalational halothane, via nose cone, then fixed in a sterotaxic frame. The dorsal spinal cord in the region T8-T12 was exposed by cutting the lateral processes of the vertebra. A reproducible hemisection of the dorsal two-thirds of the spinal column was made using a razor blade fragments attached to the pivot point of cut down micro-scissors fixed in the electrode holder of the stereotaxic frame. Bleeding was stemmed using absorbent surgical gauze and a piece of artificial dura (Dura-film, UpJohn ˜1.5×3 mm) was placed over the incision in the spinal cord. Sutures (5/0) were used to close the separated muscles and cutaneous incision. Lesion completeness was determined by absence of anterograde tracer in the CST caudal to the injury site in parallel animals.

To prepare the sural nerve for subsequent use as graft tissue, the left sural nerve was exposed and ligated with 5/0 suture via a 1 cm incision in the lower thigh, 1 week prior to grafting. At the time of grafting, the preligated sural nerve distal to the ligation was removed and the spinal cord injury cavity exposed. In some animals, a 3-5 mm piece of preligated sural nerve was inserted into the injury cavity by attaching a 10/0 suture to the graft tissue, passing the suture needle though the cavity and then drawing the nerve tissue into the cavity. The space remaining in the cavity was filled with 1-3 μl of gelling collagen solution in DMEM containing a final concentration of 50 μg/ml each of BDNF, NT-3 and GDNF. In other animals, the preligated sural nerve was removed to a sterile microfuge tube and minced with micro-scissors, then ice cold gelling collagen-solution and neurotrophic factor cocktail added, drawn up into a 10 μl Hamilton syringe and immediately injected into the cavity. Animals were allowed to survive for 2 weeks before being processed for histology: Two days prior to sacrifice, dried mixtures of wheat germ agglutinin-conjugated horseradish peroxidase (WGA-HRP; Vector Laboratories, Buringame, Calif., USA) and coated with horse radish peroxidase (HRP; Sigma, St Louis, Mo., USA) were implanted into the sensory motor cortex. Forty eight hours later, animals were perfusion fixed using 4% paraformaldehyde, 0.5% glutaraldehyde in 0.1 M phosphate buffer at pH 7.3. The whole central nervous system was dissected out, cryoprotected in 30% sucrose and serial coronal 50 μm frozen sections cut and thaw mounted on 4% gelatin coated slides. Tetramethybenzidine (TMB; Sigma, St Louis, Mo., USA) was used to visualise the HRP-labelled CST processes. The number of individual labelled CST processes were counted and distances from the rostral end of the cavity wall measured.

### EXAMPLE II

Monitoring of CST Regeneration Using Biotinylated Dextran as Neuronal Tracer

The procedure was essentially as described in Example 1, with the following changes: Animals were allowed to survive for 12 months before being processed for histology following anterograde labelling using biotinyated dextran as the neuronal tracer: Two weeks prior to sacrifice, dried biotinylated-dextran (BDA, 10,000 MW, lysine fixable; Molecular Probes, Eugene, Oreg., USA) was implanted into the sensory motor cortex. Fourteen days later, animals were perfusion fixed using 4% paraformaldehyde, 0.5% glutaraldehyde in 0.1 M phosphate buffer at pH 7.3. The whole central nervous system was dissected out, cryoprotected in 30% sucrose and serial coronal 50 μm horizontal sections cut and thaw mounted on 4% gelatin coated slides. Tetramethybenzidine (TMB; Sigma, St Louis, Mo., USA) was used to visualise the HRP-labelled CST processes. Camera lucida drawings were made of the injury site and photomicrographs taken of BDA labelled CST processes that had grown through the cavity walls and beyond the injury site.

### EXAMPLE III

Infusion of NT-3 Followed by Grafting of a Suspension of Pre-Ligated Peripheral Nerve Tissue into Spinal Cord Injury Cavity

The procedure was essentially as described in Example 1, with the following changes: A keyhole surgical approach was used to gain access to the spinal cord. In this approach, the muscles overlaying the T10 vertebrae were separated by blunt dissection and a 2-3 mm² window in the dorsal vertebra was created using bone rogues to gain access to the underlying spinal cord. A dorsal hemisection of the spinal cord was performed as described in Example 1. One week prior to grafting while the animals was anaesthetised for sural nerve ligation, the spinal cord injury cavity was re-exposed and a length of 27 gauge silicone tubing was inserted into the cavity and connected to a subcutaneously implanted osmotic minipump (Model 1007D, Alza, Palo Alto, Calif.) containing 50 μg/ml NT-3 in DMEM or DMEM alone. This operation was performed at the time of sural nerve ligation. Following infusion into the cavity of DMEM with or without NT-3 at a rate of 0.5 μl/hr for 1 week, the catheter was cut, the osmotic pump removed and a suspension of minced preligated sural nerve in ice cold gelling collagen solution (without neurotrophic factors) was injected into the cavity via the pump catheter. The CST was anterogradely labelled using BDA and the procedure described in Example II. The animals were allowed to survive for 2 weeks before being processed for histology.

### EXAMPLE IV

Injection or Infusion of NT-3 Spinal Cord Injury Cavity to Identify Therapeutic Agents Able to Stimulate Regrowth of the Chronically Injured CST

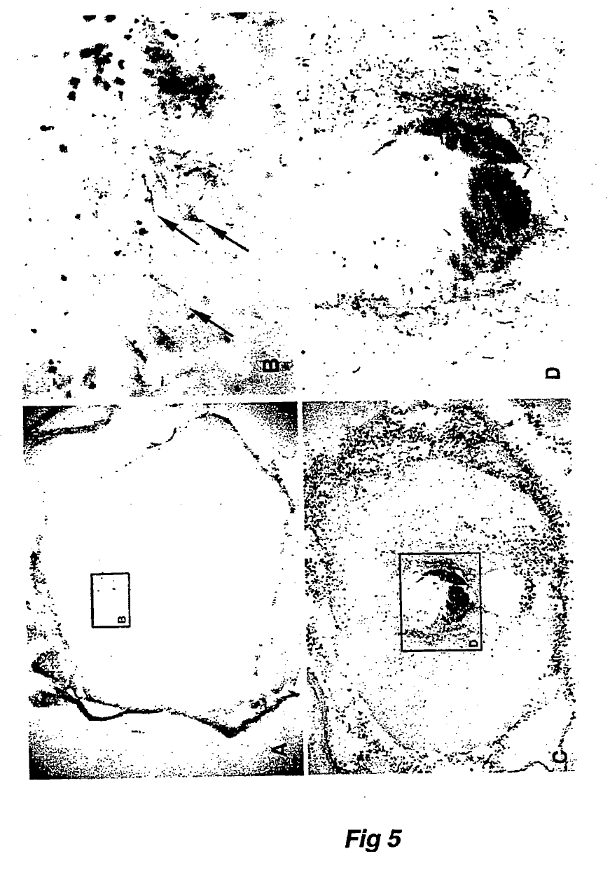

The procedure was essentially as described in Example III except that no peripheral nerve graft tissue was injected into the spinal cord injury cavity after injection or infusion of NT-3 and that BDA or HRP/WGA-HRP was used as the neuronal tracer for anterograde labelling of the CST. As shown in FIG. 5, infusion of NT-3 stimulated sprouting of the CST. No sustained regeneration of CST sprouting was observed as evidenced by absence of any labelled processes more than 2 mm distal to the rostral end of the cavity 12 or 17 weeks later.

### EXAMPLE V

Grafting of a Suspension of Pre-Ligated Peripheral Nerve Tissue into Spinal Cord Injury Cavity Due to Compression Injury

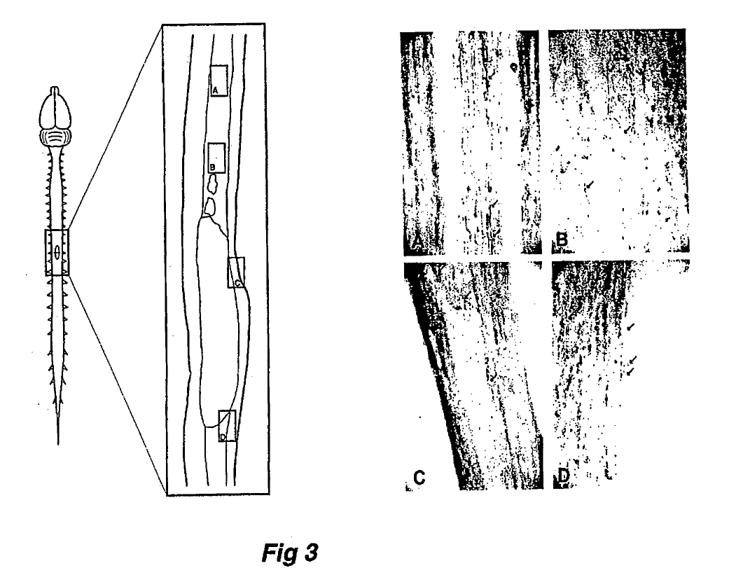

The procedure was essential as described in Example I except that: minced preligated saphenous nerve instead of minced preligated sural nerve was used to prepare the graft tissue for injection. The saphenous nerve was ligated via 1 cm incision in the upper third of left thigh and left for 1 week prior to removal. The graft tissue was injected into spinal cord injury cavity that had developed 3 weeks after weight drop spinal cord injury. This injury was generated by positioning a custom made apparatus modelled on the New York University spinal cord injury devise. It was positioned over the exposed spinal cord, with the teflon probe resting gently on the dorsal surface of the spinal cord. The teflon weight of 10 gm was allowed to falls under gravity down a vertical rod a distance of 1.25 cm and impact on top of the probe resting on the surface of the spinal cord, thus briefly compressing the spinal cord. The histological processing of the animals was essentially as used in Example II and demonstrated CST sprouting and regeneration through the grey matter of the cavity walls (result not shown but similar to that illustrated in FIG. 3).

By using the examples described above, results may be generated to demonstrate that the chronically injured CST can be stimulated to grow past the spinal cord injury site by administering into the spinal cord injury cavity in vivo activated glial cell based tissues or injectable formulations derived from the tissues.

In animal models of acute spinal cord injury (SCI) to date, the injured CST can not be easily stimulated to regenerate. Typically, as a natural consequence of spinal cord injury, a cavity forms. This naturally occurring cavity can then be used as a depository for therapeutic agents to provide a clinically useful approach for the delivery of nerve growth promoting agents to promote regeneration of the CST as part of a course of treatment for chronic spinal cord injury.

A number of tissues or cell grafts are currently used to stimulate axonal sprouting of acutely injured CST. A problem sometimes associated with the use of tissues or cell grafts is that they may be subject to immunological rejection. The utilisation of tissue or cell grafts from an autologous source is useful for overcoming immunological rejection problems in addition to stimulating CST regeneration. The source of nerve material containing in vivo activated glial cells for use in the nerve grafting procedures is in preference, but not limited to, selected from the sural or saphenous nerves, olfactory epithelium or stem cells. In addition nerve growth stimulating molecules such as neurotrophic factors may also be added.

1. A nerve growth promoting material having as an active agent, material derived from nerve material that has been separated from a nerve, within a living mammalian body, which has had some of the functional connection between the nerve cell bodies and the nerve material previously interrupted for a substantial period of time.

2. A nerve growth promoting material as in claim 1 wherein said substantial time is at least 48 hours.

3. A nerve growth promoting material as in claim 1 further characterised in that said interruption is as a result of ligation of said nerve material.

4. A nerve growth promoting material as in claim 1 further characterised in that said interruption is as a result of severing said nerve material.

5. A nerve growth promoting material as in claim 1 further characterised in that said interruption is as a result of crushing said nerve material.

6. A nerve growth promoting material as in claim 1 further characterised in that the interruption is effected by functioning nerve material being treated with agents which cause the nerve axons to degenerate.

7. A nerve growth promoting material as in any one of the preceding claims wherein said nerve material includes non-functioning nerve axons.

8. A nerve growth promoting material as in claim 1 wherein said substantial time is within the period of from 2 to 10 days.

9. A nerve growth promoting material as in any one of the preceding claims further characterised in that the active agent is in combination with a physiologically acceptable support matrix.

10. A nerve growth promoting material as in claim 9 wherein said physiologically acceptable support matrix is collagen.

11. A nerve growth promoting material as in claim 9 wherein said physiologically acceptable support matrix is fibrin.

12. A nerve growth promoting material as in any one of the preceding claims further characterised in that the mammalian body is human.

13. A nerve growth promoting material as in any one of the preceding claims further characterised in that said nerve material is abundant with non-neuronal cells.

14. A nerve growth promoting material as in any one of the preceding claims further characterised in that said nerve material is abundant with axon myelinating cells.

15. A nerve growth promoting material as in any one of the preceding claims further characterised in that said nerve material is abundant with in vivo activated glial cells.

16. A nerve material as in any one of claims 13 to 15 further characterised in that said nerve material is derived from the sural nerve.

17. A nerve material as in any one of claims 13 to 15 further characterised in that said nerve material is derived from the olfactory epithelium.

18. A nerve material as in any one of claims 13 to 15 further characterised in that said nerve material is derived from the saphenous nerve.

19. A nerve growth promoting material as in claim 1 that further includes material derived from stem cells.

20. A nerve growth promoting material as in claim 1 that includes growth promoting molecules effective to stimulate a chronically injured corticospinal tract to commence re-growing.

21. A nerve growth promoting material as in claim 20 wherein said growth promoting molecules are neurotrophic factors.

22. A nerve growth promoting material as in claim 21 wherein said growth promoting molecules are selected to be those which act on the neurotrophin receptors, trkB or trkC.

23. A nerve growth promoting material as in any one of the preceding claims 1 to 20 further characterised in that said nerve material is for use in a host and contains only cells of identical DNA to the DNA of the intended host.

24. A nerve growth promoting material resulting from a nerve that has had the functional flow of the axons interrupted therefrom after a substantial period of time.

25. A nerve growth promoting material resulting from a nerve that has had the functional flow of the axons interrupted therefrom after a substantial period of time for use for insertion into an injury cavity of the central nervous system.

26. A nerve growth promoting material resulting from a nerve that has had the functional flow of the axons interrupted therefrom after a substantial time for use for insertion into an injury cavity of a central nervous system in the vicinity of a nerve the growth of which is to be promoted.

27. A method of promoting nerve growth which method includes the steps of interrupting the functional flow of the axons in a nerve in a living mammalian body, separating the injured material after a substantial time, minutely dividing the material, combining said material with a physiologically acceptable support matrix, and inserting said mixture into a cavity in close proximity to a nerve ending the growth of which is to be assisted within a central nervous system of a living mammalian body.

28. A method of promoting nerve growth which method includes the steps of interrupting the functional flow of the axons in a nerve in a living mammalian body, separating the injured material after a substantial time, minutely dividing the material, combining said material with a physiologically acceptable support matrix, and inserting said mixture into a cavity adjacent a nerve ending the growth of which is to be assisted within a central nervous system and where there is grey matter for the nerve to grow around or through.

29. A method of promoting nerve growth as in preceding claim 27 or 28 further characterised in that the material is inserted into a central nervous system which is of a body which has an identical DNA to the body from which the material was derived.

30. A method of assisting nerve growth as in preceding claim 27, 28 or 29 further characterised in that the material is inserted into a central nervous system which is of a body which is the same body from which the material was derived.

31. A method of promoting nerve growth as in any one of the preceding claims 27 to 30 wherein said nerve growth promoting material in combination with physiologically acceptable support matrix is introduced into a cavity or space within the central nervous system.

32. The nerve growth material as in any one of the preceding claims 1 to 26 when in a cavity of a central nervous system of a living mammalian body.

33. A method of promoting nerve growth as in any one of the preceding claims 27 to 30 in a subject having nerve damage as a result of physical or surgical trauma, bacterial or viral infection, Parkinson's disease or Alzheimer's disease.

34. A method of promoting nerve growth as in any one of the preceding claims 27 to 30 in an animal having peripheral nerve damage as a result of physical or surgical trauma, bacterial or viral infection, or degenerative disease.

35 A nerve growth promoting material as in claim 1 wherein said substantial time is within the period of from 2 to 60 days.

36 A nerve growth promoting material as in claim 1 wherein said substantial time is approximately 7 days.

37 A nerve growth promoting material as in claim 1 characterised in that it contains cells modified to express the growth promoting molecules as in claim 2

38 A method of promoting nerve growth which includes the steps of locating a nerve growth promoting material, as in any one of the preceding claims 1 to 26, in a chronically injured CST of a living mammalian body.

39 A method of promoting nerve growth as in claim 38 in which the living mammalian body is human.

40 A method of promoting nerve growth as in either one of claims 38 or 39 further characterised in that the location of the cavity is adjacent nerve endings to be grown.

41 A method of promoting nerve growth as in any one of claims 38, 39 or 40 further characterised in that the location of the cavity is adjacent grey matter around which regrowth may occur.

42 A method of promoting nerve growth as in any one of claims 38, 39, 40 or 41 further characterised in that the location of the cavity is adjacent nerve endings to be grown.
